(19) United States
(12) Patent Application Publication
(10) Pub. No.: US 2024/0374335 A1
(43) Pub. Date: Nov. 14, 2024

(54) SURGICAL INCISE DRAPE

(71) Applicant: 3M INNOVATIVE PROPERTIES COMPANY, St, Paul, MN (US)

(72) Inventors: Assumpta A.G. BENNAARS-EIDEN, Andover, MN (US); Philip N. STANKARD, Minneapolis, MN (US); Joanna Esther Z. SCHNEIDER, Stillwater, MN (US); Diane L. KOCH, St. Paul, MN (US); John W. SPERLING, Rochester, MN (US); Richard L. JACOBSON, Stillwater, MN (US)

(21) Appl. No.: 18/552,947

(22) PCT Filed: Apr. 1, 2022
(86) PCT No.: PCT/IB2022/053083

§ 371 Date: Sep. 28, 2023

Related U.S. Application Data
(60) Provisional application No. 63/169,990, filed on Apr. 2, 2021.

(51) Int. Cl. A61B 46/20

(52) U.S. Cl. CPC A61B 46/20; A61B 2046/201; A61B 2046/205

(57) ABSTRACT

Trapezoidal surgical incise drapes designed for shoulder surgery are described. Also described are methods of preparing trapezoidal surgical incise drapes and methods of using trapezoidal surgical incise drapes for shoulder surgery.

### BACKGROUND

Surgical-site infections often lead to prolonged hospitalizations and are heavily associated with high morbidity and mortality. A patient's risk for surgical-site infection is largely proportional to the residual bacteria at, or in proximity to, the surgical site. The steady increase in antibiotic resistance makes pre-operative efforts to reduce bacteria density at surgical sites crucial.

Disinfecting procedures reduce the risk of infection, as does covering the surgical site with a surgical incise drape having an antimicrobial-impregnated adhesive. However, contoured areas are difficult to adequately drape since creasing or wrinkling of the surgical incise drape creates a gap between the skin and the drape. Gaps prevent thorough and continued disinfection and may allow for further bacterial introduction.

Shoulder operations are particularly susceptible to infection due to the difficulty in draping a patient's arm. Standard surgical incise drapes are square or rectangular in shape and often wrinkle, crease, or otherwise fold onto themselves when trying to drape portions of the arm.

What is needed is a surgical incise drape or set of or surgical incise drapes in a shape that is capable of covering the contours of the arm.

### SUMMARY

In one embodiment, a surgical incise drape is described. The surgical incise drape includes a flexible substrate having a trapezoidal configuration characterized by four vertices A, B, C, and D; and an antimicrobial adhesive composition. The flexible substrate includes a first base (AB) defined by a first base length (ABL) measured between vertices A and B; a second base (CD) defined by a second base length (CD_{L}) measured between vertices C and D, wherein AB_{L}>CD_{L}; a first side (AC) defined by a first side length (AC_{L}) measured between vertices A and C; and a second side (BD) defined by a second side length (BD_{L}) measured between vertices B and D. The antimicrobial adhesive composition includes an antimicrobial agent and a pressure-sensitive adhesive.

In one embodiment, a surgical incise drape is described. The surgical incise drape includes a flexible substrate divided into three separate subsections, wherein the three separate sections together form a trapezoidal configuration characterized by four vertices A, B, C, and D; and an antimicrobial adhesive composition. The flexible substrate includes a first base (AB) defined by a first base length (AB_{L}) measured between vertices A and B; a second base (CD) defined by a second base length (CD_{L}) measured between vertices C and D, wherein AB_{L}>CD_{L}; a first side (AC) defined by a first side length (AC_{L}) measured between vertices A and C; and a second side (BD) defined by a second side length (BD_{L}) measured between vertices B and D. The antimicrobial adhesive composition includes an antimicrobial agent and a pressure-sensitive adhesive.

In one embodiment, a method of preparing a surgical incise drape is described. The method includes providing a flexible substrate and an antimicrobial adhesive composition; and disposing the antimicrobial adhesive composition onto the flexible substrate.

In one embodiment, a method for preparing a shoulder for surgery is described. The method includes providing a surgical incise drape described herein; and contacting the surgical incise drape to a skin surface of a subject.

In one embodiment, a kit is described. The kit includes one or more surgical incise drapes described herein; and a set of instructions directed a user to perform method steps for preparing a shoulder for surgery.

### BRIEF DESCRIPTION OF DRAWINGS

FIG. 1A illustrates a surgical incise drape of the present disclosure.

FIG. 1B illustrates a surgical incise drape of the present disclosure having contouring.

FIG. 1C illustrates a surgical incise drape of the present disclosure having a delivery system.

FIG. 1D illustrates a surgical incise drape of the present disclosure having a delivery system with handling tabs.

FIG. 1E illustrates a surgical incise drape of the present disclosure having a delivery system with handling tabs.

FIG. 1F illustrates surgical incise drape of the present disclosure having contouring and a delivery system.

FIG. 2A illustrates a surgical incise drape of the present disclosure.

FIG. 2B illustrates a surgical incise drape of the present disclosure having contouring.

FIG. 2C illustrates a surgical incise drape of the present disclosure having a delivery system.

FIG. 2D illustrates a surgical incise drape of the present disclosure having subsection contouring.

FIG. 2E illustrates a separated three-piece surgical incise drape of the present disclosure having subsection contouring.

FIG. 3 illustrates a surgical incise drape of the present disclosure overlaying a subject's arm.

FIG. 4A illustrates a cross-sectional view of a surgical incise drape of the present disclosure.

FIG. 4B illustrates a cross-sectional view of a surgical incise drape of the present disclosure.

FIG. 4C illustrates a cross-sectional view of a surgical incise drape of the present disclosure.

FIG. 5 is a flow chart of a method for preparing a surgical incise drape of the present disclosure.

FIG. 6 is a flow chart of a method for preparing a subject's shoulder for surgery.

FIG. 7 illustrates a kit including a surgical incise drape of the present disclosure.

### DETAILED DESCRIPTION

Propionibacterium acnes commonly colonizes the skin, among other infectious organisms, and is responsible for more than half of the post-operative infections involving the shoulder. Several technological advances have limited surgical site infections, most notably the use of surgical incise drapes, however it is challenging to drape contoured areas of the body (e.g., shoulder), which is largely due to wrinkling of the drape. When a surgical incise drape wrinkles, areas beneath the wrinkles are not adequately disinfected, thereby leading to an increase in chance for infection.

The surgical incise drapes of the present disclosure directly address the challenges of draping the shoulder and surrounding areas. The trapezoidal configuration serves to limit or eliminate wrinkling of the drape upon application to the skin surface. Additional features of the drape construction, e.g., contouring, delivery systems, and drape sectioning, further assist in application of the drape and assure greater skin contact. Delivery systems provide structural reinforcement which can prevent drapes from creasing or otherwise folding onto themselves during application. Moreover, the delivery systems of the present disclosure may be removed in portions, thus allowing piecewise application of the drape meanwhile maintaining reinforcement for the remainder of the drape. In addition to greater application control, three-part drape sectioning (i.e., drape seal, incise seal, and stockinette seal) would allow for surgeons to modify the drape in view of the type of surgery. For example, anthroscopic surgery may only require a drape seal and a stockinette seal as compared to open surgery requiring the full drape.

### Definitions

As used herein, “about” means ±10 percent of a given value. For example, about 10 means 9 to 11.

As used herein, “contouring” refers to a structural impression that deviates from the overall trapezoidal configuration. All non-straight features are considered contouring. Contouring allows for the surgical incise drapes of the present disclosure to be more readily placed around body structures. “Concave contouring” refers to surface area removed from the trapezoid configuration defined by vertices A, B, C, and D, or surface are removed from subsectional areas (i.e., subsection seals). “Convex contouring” refers to surface area added to the overall trapezoid configuration, i.e., surface area extending beyond the trapezoid configuration defined by vertices A, B, C, and D, or surface area added to subsectional areas. Contouring may prevent wrinkling.

As used herein, “disinfecting” refers to a reduction in the number of active microorganisms present on a surface being disinfected. Disinfecting may kill or prevent microorganisms from growing or proliferating.

As used herein, “plasticizer” refers to a substance or combination of substances that lowers the glass transition temperature of another substance (e.g., a pressure-sensitive adhesive). Plasticizers effectively soften, increase flexibility, increase plasticity, decrease viscosity, and/or decrease friction of a substance to which it is added. The plasticizers described herein are relatively low molecular weight polymers that increase the spacing between chains of a larger molecular weight polymer (i.e., PSA).

As used herein, “polymer” refers to a substance having one or more repeating monomer units. The chemical identities of the polymeric substances herein are at times described in terms of the monomers to which the polymer is derived. A skilled artisan would readily understand the reactivity profile of the recited monomers and how the monomers could synthetically be joined to form the polymer.

As used herein, “pressure-sensitive adhesive” refers to a non-reactive, self-stick adhesive that forms a bond when pressure is applied. No solvent, water, or heat is required to activate a pressure-sensitive adhesive.

When referring to “solubility,” or “to solubilize” it should be understood that the solubility of a component A in a component B refers to conditions in which only component A and component B are present, e.g., no added salts, compounds, or the like. Furthermore, any solubility values provided herein are with regard to a temperature range of about 20° C. to about 23° C. at atmospheric pressure (i.e., 760 mm/Hg).

As used herein, “trapezoid” refers to a shape consisting of four vertices joined by straight lines. Two lines join to form a vertex at an angle. No more than two angles have the same value.

As used herein, “vertices A and B” are determined to be the points defining the greatest width distance (i.e., ABL) within the flexible substrate. “Vertices C and D” are determined to be the points defining the least width distance (i.e., CD_{L}) within the flexible substrate. Any surface area extending beyond the vertices is considered contouring.

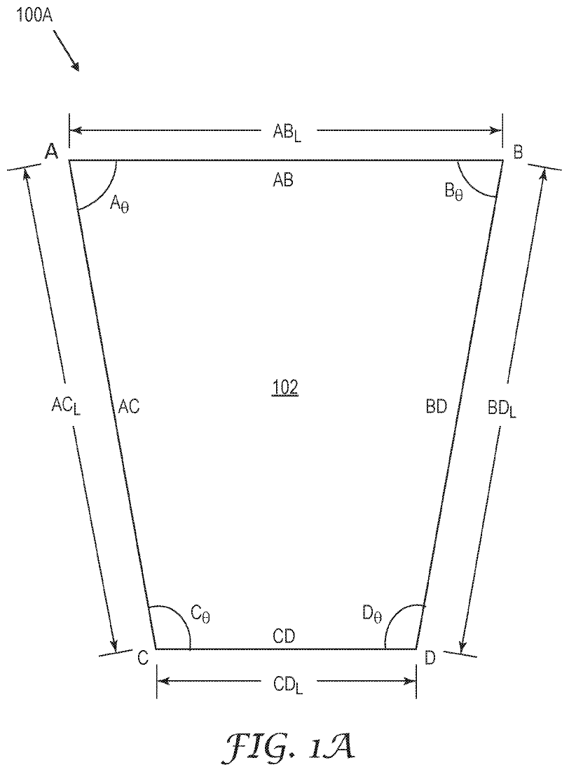

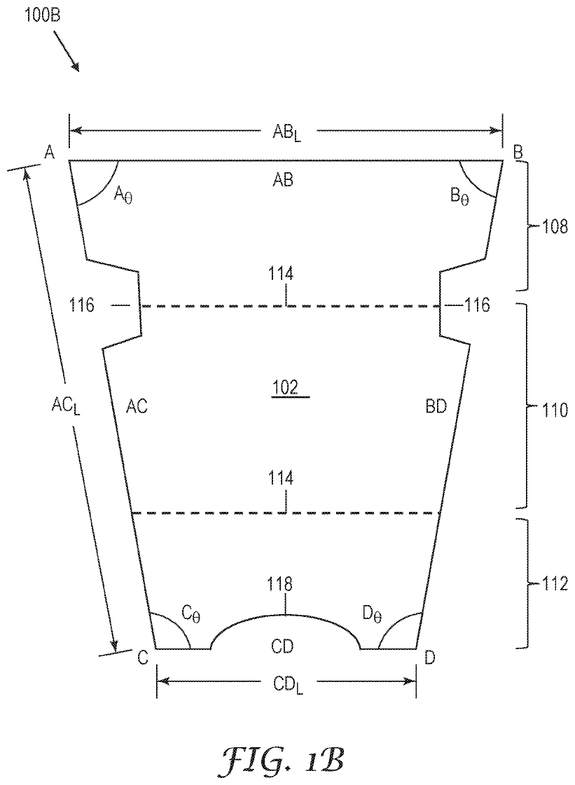

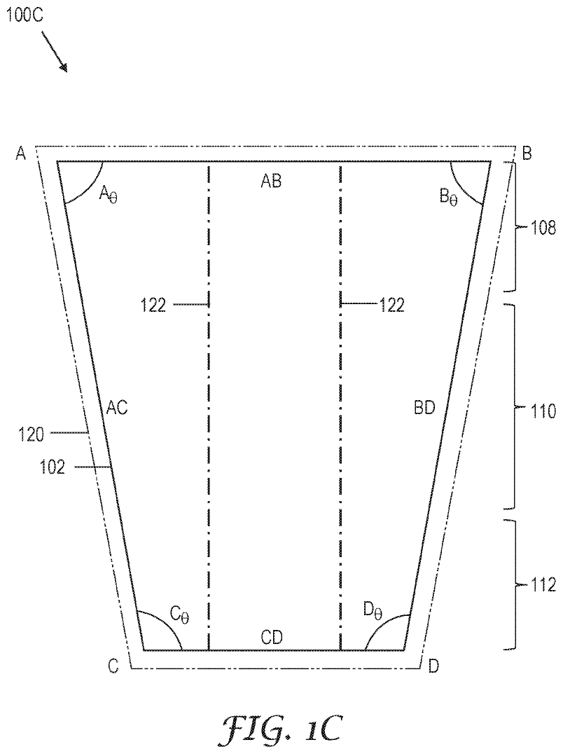

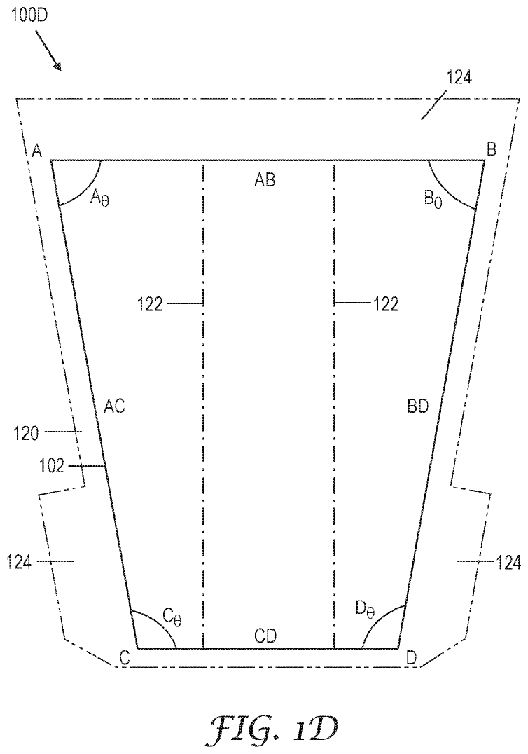

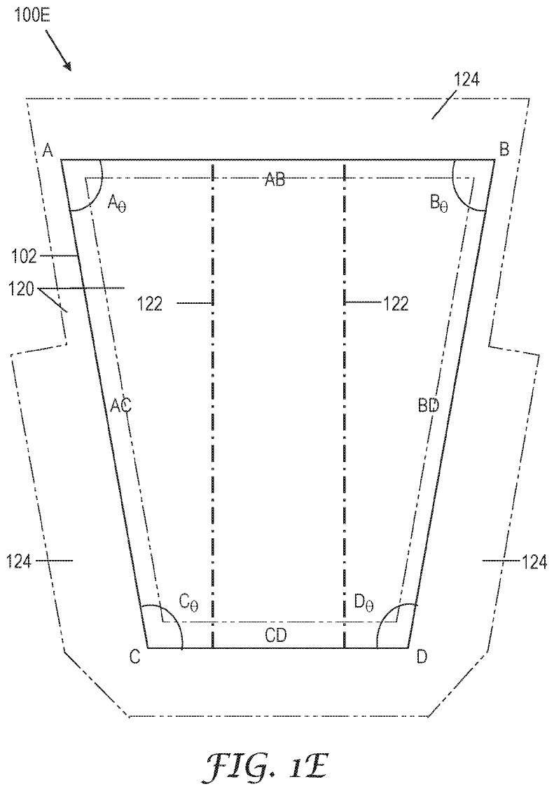

FIG. 1A depicts a one-piece trapezoidal surgical incise drape 100A of the present disclosure. Surgical incise drape 100 includes a flexible substrate 102 having a first base AB defined by a first base length AB as measured between vertices A and B, a second base CD defined by a second base length CD_{L} as measured between vertices C and D, a first side AC defined by a first side length AC_{L} as measured between vertices A and C, and a second side BD defined by a second side length BD_{L} as measured between vertices B and D. As depicted, first length AB is greater than second length CD_{L}. First base AB and first side AC join at vertex A at an angle A_{θ}. First base AB and second side BD join at vertex B at an angle B_{θ}. Second base CD and first side AC join at vertex C at an angle C_{θ}. Second base CD and second side BD join at vertex D at an angle D_{θ}. Surgical incise drape 100A further includes an antimicrobial adhesive composition (not shown) having an antimicrobial agent and a pressure-sensitive adhesive contacting a major surface of the flexible substrate 202.

FIG. 1B depicts a one-piece trapezoidal surgical incise drape 100B having all the features of surgical incise drape 100A. All surgical incise drapes of the present disclosure include areas configured to drape sections of a subject's arm in preparation for surgery. These areas are referred to flexible substrate subsections. As depicted, the flexible substrate subsections include: a drape seal 108, an incise site seal 110, and a stockinette seal 112. Drape seal 108 configured to overlay the skin surfaces above the proximal end of a humerus, the distal end of a clavicle, and at least a portion of the neck that is typically covered in nonwoven drape material. Drape seal 106 could adhere to nonwoven drape material under current surgical practices. Incise site seal 110 is configured to overlay the shoulder, bicep, tricep, and wrap to cover the under-armpit area. Stockinette seal 112 is configured to overlay and at least partially encircle the forearm. As depicted, flexible substrate 102 includes printed markings 114. Flexible substrate 102 is further shown to include side contouring 116 and base contouring 118. Contouring may assist in the application of surgical incise drape 100B and prevent wrinkling of flexible substrate 102.

FIG. 1C depicts a one-piece trapezoidal surgical incise drape 100C having all the features of surgical incise drape 100A. Surgical incise drape 100C further includes a delivery system 120. As depicted, delivery system 120 is in the form of a surface liner covering the entire area of flexible substrate 102. In this particular illustration, delivery system 120 includes two cuts 122 that separate the delivery system into three liner sections, in which the liner sections may be removed independently. A sectioned delivery system may allow for easier application of surgical incise drape 100C and prevent wrinkling.

FIG. 1D depicts a one-piece trapezoidal surgical incise drape 100D having all the features of surgical incise drape 100C. Delivery system 120, as shown, includes handling tabs 124. Handling tabs 124 may further assist in application of surgical incise drape 100D.

FIG. 1E depicts a one-piece trapezoidal surgical incise drape 100E having all the features of surgical incise drape 100D. Delivery system 120, as shown, includes two liners—a surface area liner and a perimeter liner. The perimeter liner is ideally constructed from a material more rigid than a material suitable for a surface liner. The perimeter liner may aid in handling of surgical incise drape 100E, e.g., preventing the drape from folding onto itself during handling.

FIG. 1F depicts a one-piece trapezoidal surgical incise drape 100F. Surgical incise drape 100F is shown to include a flexible substrate 102 having contouring 126 along first base AB and contouring 128 along first side AC and second side BD. Contouring 128 resides between drape seal 108 and surgical incise seal 110. Surgical incise drape 100F is further shown to include a delivery system 120 having a surface area liner 121 and a perimeter liner 123 (including handling tabs 124). Surface liner 121 includes cut lines 114 (printing markings or perforations) in which a user may cut along said lines to form a three-piece drape. Surface liner 121 further includes perforation 122 for sectional removal of the liner, and guide marking 115 for aiding in proper placement of drape seal 108.

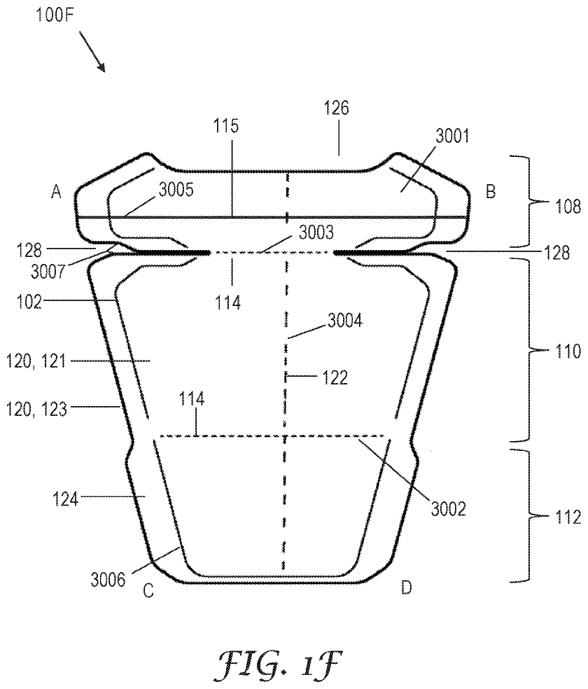

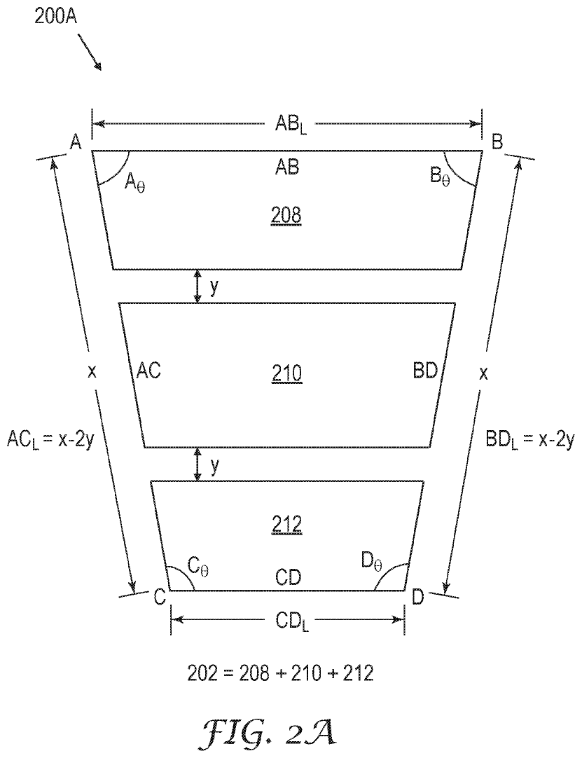

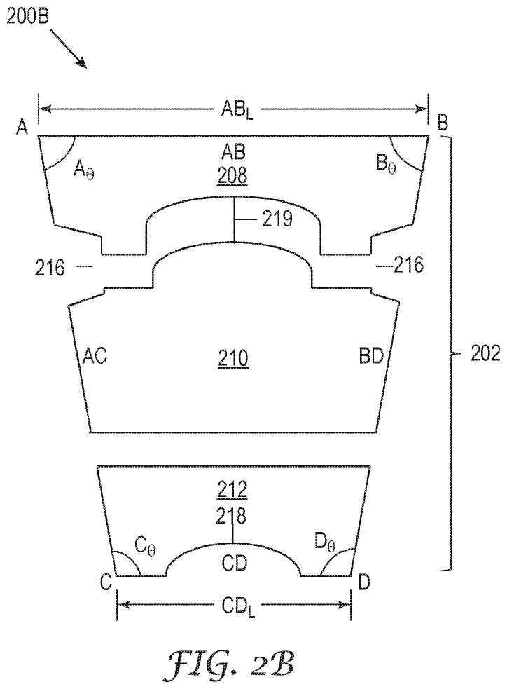

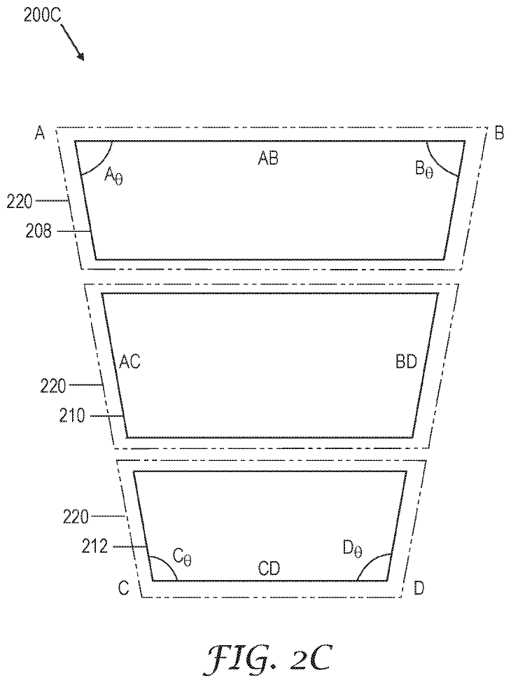

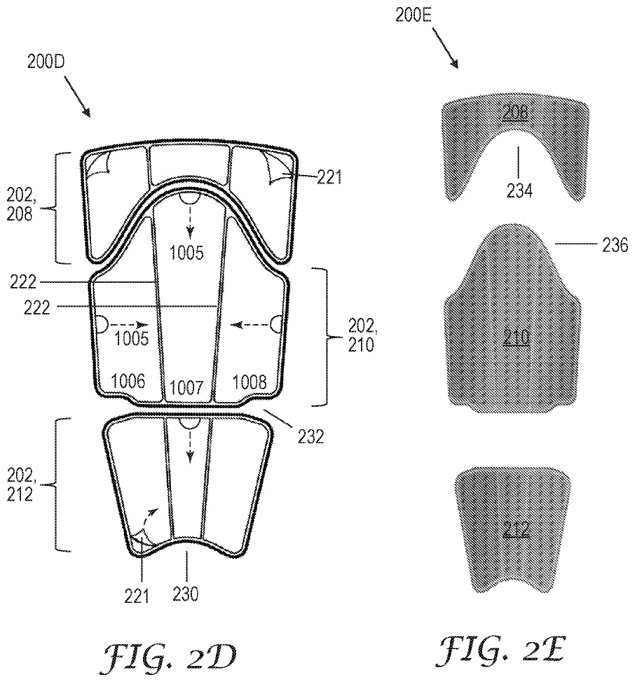

FIG. 2A depicts a three-piece trapezoidal surgical incise drape 200A of the present disclosure. Surgical incise drape 200A includes all the features of surgical incise drape 100A, including three flexible substrate subsections 208 (i.e., drape seal), 210 (i.e., incise site seal), and 212 (i.e., stockinette seal) that when nested together form flexible substrate 202 having a trapezoidal configuration.

FIG. 2B depicts a three-piece trapezoidal surgical incise drape 200B of the present disclosure having all the features of surgical incise drape 200A. Flexible substrate 202 is further shown to include side contouring 216, base contouring 218, and subsection contouring 219. Contouring may assist in the application of surgical incise drape 100B and prevent wrinkling of flexible substrate 102. For example, subsection contouring, such as 219, may allow for greater precision in applying subsections 208 and 210 around a shoulder area.

FIG. 2C depicts a three-piece trapezoidal surgical incise drape 200C of the present disclosure having all the features of surgical incise drape 200A. Surgical incise drape 200C is shown further including a delivery system 320 (surface liner) on each of the flexible substrate subsections 308, 310, and 312. Although not shown, delivery system 320 may further include handling tabs and/or a perimeter liner on one or more of the flexible substrate subsections.

FIG. 2D depicts a three-piece trapezoidal surgical incise drape 200D of the present disclosure. Surgical incise drape 200D is shown to include a flexible substrate 202 (subsections: drape seal 208, surgical incise seal 210, and stockinette seal 212) having second base CD contouring 230 and first side AC and second side BD contouring 232. A delivery system (i.e., surface area liner 221) is shown having two liner perforations 222 for sectioned removal.

FIG. 2E depicts three-piece trapezoidal surgical incise drape 200E wherein flexible substrate subsections 208, 210, and 212 are separated. As shown drape seal 208 includes concave contouring 234 and surgical incise seal 210 includes convex contouring 236, wherein contouring 234 and 236 are configured to nest together. This particular contouring pattern may allow for better coverage of the shoulder region.

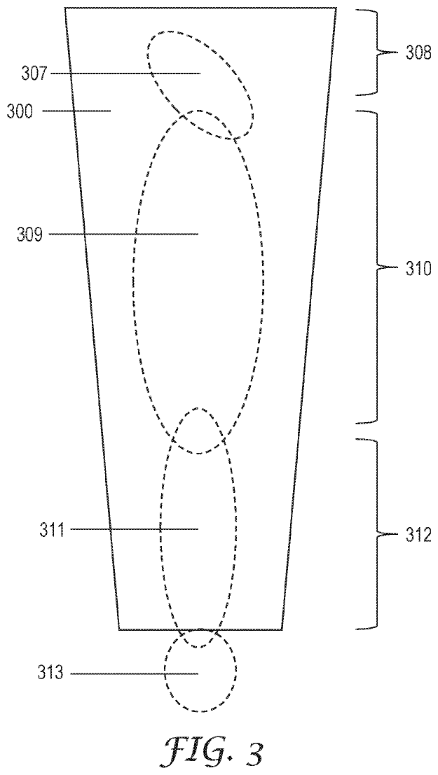

FIG. 3 depicts a trapezoidal surgical incise drape 300 of the present disclosure overlaying a subject's arm. The subject's arm is divided into four sections: a neck/clavicle region 307, a shoulder/upper arm region 309, a forearm region 311, and a hand region 313. Surgical incise drape 300 is shown to include three flexible substrate subsections: drape seal 308, surgical incise seal 310, and stockinette seal 312. Although shown as a one-piece construction, one can easily envision the flexible substrate subsections separated into a three-piece constructions and nested in the trapezoidal configuration.

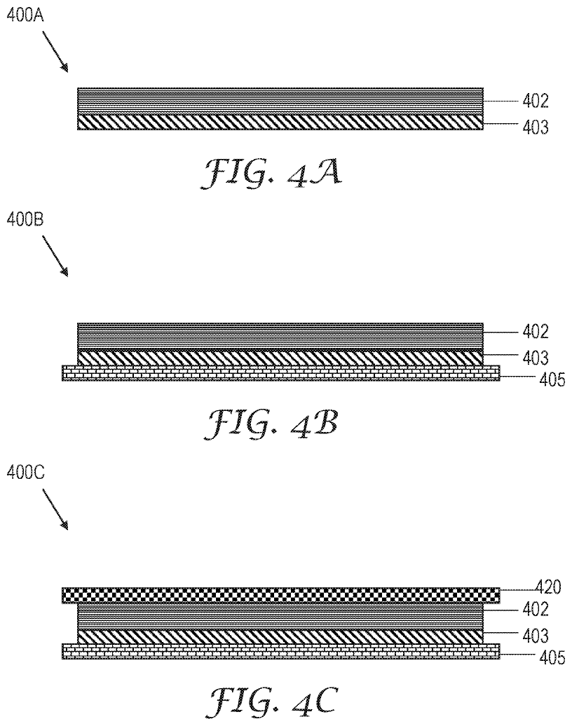

FIG. 4A depicts a cross-sectional view of a trapezoidal surgical incise drape 400A described herein. Surgical incise drape 400A is shown having a flexible substrate 402 and an antimicrobial adhesive composition 403 in contact with flexible substrate 402.

FIG. 4B depicts a cross-sectional view of a trapezoidal surgical incise drape 400B described herein. Surgical incise drape 400B is shown having a flexible substrate 402 and an antimicrobial adhesive composition 403 in contact with flexible substrate 402, and a release liner 305 in contact with antimicrobial adhesive composition 403.

FIG. 4C depicts a cross-sectional view of a trapezoidal surgical incise drape 400C described herein. Surgical incise drape 400C is shown having a flexible substrate 402 and an antimicrobial adhesive composition 403 in contact with flexible substrate 402, a release liner 405 in contact with antimicrobial adhesive composition 403, and a delivery system 420 in contact with flexible substrate 402.

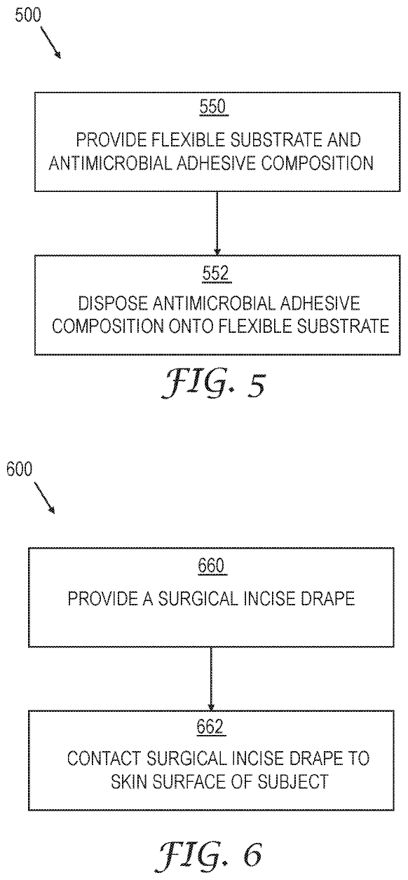

FIG. 5 is a flow chart representing a method 500 for preparing a surgical incise drape of the present disclosure. The method includes 550 providing a flexible substrate and an antimicrobial adhesive composition and 552 disposing the antimicrobial adhesive composition onto a major surface of the flexible substrate.

FIG. 6 is a flow chart representing a method 600 for preparing a shoulder for surgery. The method includes 660 providing a surgical incise drape of the present disclosure and 662 contacting the surgical incise drape to a skin surface of a subject.

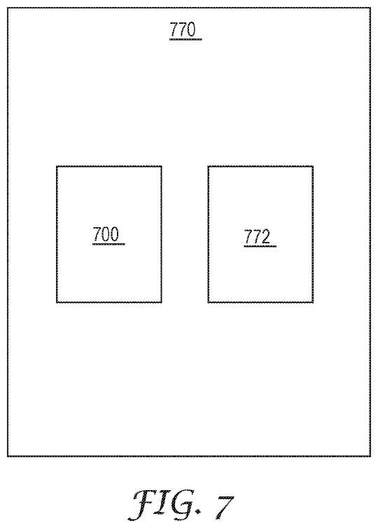

FIG. 7 depicts a kit 770 including one or more surgical incise drape 700 of the present disclosure and a set of instructions 772 directing a user to contact surgical incise drape 700 to a skin surface of a subject.

### Surgical Incise Drapes

(One-piece drape) In various embodiments, a surgical incise drape is described. The surgical incise drape may include a flexible substrate having a trapezoidal configuration characterized by four vertices A, B, C, and D; and an antimicrobial adhesive composition. The flexible substrate may include a first base (AB) defined by a first base length (AB_{L}) measured between vertices A and B; a second base (CD) defined by a second base length (CD_{L}) measured between vertices C and D, wherein AB_{L}>CD_{L}; a first side (AC) defined by a first side length (AC_{L}) measured between vertices A and C; and a second side (BD) defined by a second side length (BD_{L}) measured between vertices B and D. The antimicrobial adhesive composition may include an antimicrobial agent and a pressure-sensitive adhesive.

(Three-piece drape) In various embodiments, a surgical incise drape is described. The surgical incise drape may include a flexible substrate divided into three separate sections, wherein the three separate sections together form a trapezoidal configuration characterized by four vertices A, B, C, and D; and an antimicrobial adhesive composition. The flexible substrate may include a first base (AB) defined by a first base length (ABL) measured between vertices A and B; a second base (CD) defined by a second base length (CD_{L}) measured between vertices C and D, wherein AB_{L}>CD_{L}; a first side (AC) defined by a first side length (ACL) measured between vertices A and C; and a second side (BD) defined by a second side length (BD_{L}) measured between vertices B and D. The antimicrobial adhesive composition may include an antimicrobial agent and a pressure-sensitive adhesive.

In some embodiments, any surgical incise drape described herein may further include a delivery system described herein in contact with at least a portion of the flexible substrate. Variations of delivery system features are described in further detail below.

In some embodiments, any surgical incise drape described herein may further include a release liner described herein in contact with the antimicrobial adhesive composition. Variations of release liner features are described in further detail below.

In some embodiments, any surgical incise drape described herein may further include printed markings. The printed markings may serve as a cutting guide for users to customize the surgical incise drape. Printed markings may also serve as visual placement guides that help a user appropriately apply the drape to the intended area. Customizing the surgical incise drape may allow for a better fit or easier application. In some embodiments, the printed markings extend from the first side to the second side. The printed markings may serve to divide a one-piece surgical incise drape described herein into a three-piece surgical incise drape described herein.

### Flexible Substrates

In some embodiments, the flexible substrate may include a polyurethane, a polyester, a polyether block amide, a thermoplastic polyester, or a combination thereof. In some embodiments, the flexible substrate is a film, e.g., a film formed from one or more of the aforementioned polymers. In some embodiments, the flexible substrate may be transparent or translucent.

In some embodiments, the flexible substrate is characterized by maximum length (i.e., greatest distance measured between AB and CD) of about 0.5 m to about 0.7 m. For example, the maximum length may be (in meters) of about 0.5, 0.55, 0.6, 0.65, and 0.7, or a value between any of the preceding numbers, e.g., between about 0.55 and about 0.65, between about 0.6 and about 0.7, or the like.

In some embodiments, the flexible substrate may have a thickness of about 25 μm to about 200 μm. For example, the flexible substrate may have a thickness in μm of about 25, 30, 35, 40, 45, 50, 55, 60, 65, 70, 75, 80, 85, 90, 95, 100, 110, 120, 130, 140, 150, 160, 170, 180, 190, and 200, or a value within a range between any of the preceding values, e.g., between about 50 μm and about 80 μm, between about 30 μm and about 120, or the like.

In some embodiments, first base AB and second base CD are parallel with one another.

In some embodiments, the dimensions may be selected based on the arm structure size of an average human male, female, or child.

In some embodiments, the dimensions may be customized according to the arm structure size of the subject.

In some embodiments, the flexible substrate is in a one-piece configuration having three regions: a drape seal section, an incise site seal section, and a stockinette seal section. In other embodiments, the flexible substrate is in a three-piece configuration, i.e., the surgical incise drape includes three separated flexible substrate subsections, i.e., a drape seal section, an incise site seal section, and a stockinette seal section. Details of the substrate subsection are further described below.

In some embodiments, the flexible substrate may further include contouring along one or more of first base AB, second base CD, first side AC, and second side BD. Contouring makes up no greater than about 15% of the total surface are of the trapezoid. In other words, the overall area removed and/or gained by contouring can be no greater than about 15% of the trapezoid surface area defined by the area between vertices A, B, C, and D. For example, contouring may add or subtract an area of about 15%, 14%, 13%, 12%, 11%, 10%, 9%, 8%, 7%, 6%, 5%, 4%, 3%, 2%, or about 1% of the overall flexible substrate surface area. Contouring is described in more detail below.

### Characteristics of Flexible Substrate Subsections

The one-piece surgical incise drape construction and the three-piece surgical incise drape construction are designed to overlay the subject's arm extending from the neck to the wrist. The three subsections include: the drape seal, the incise site seal, and the stockinette seal.

In some embodiments, the drape seal subsection may extend from first base AB to about 20% to about 33% of the maximum length of the flexible substrate. For example, the drape seal subsection may extend from first base AB to about 20%, 21%, 22%, 23%, 24%, 25%, 26%, 27%, 28%, 29%, 30%, 31%, 32%, or 33% of the maximum length of the flexible substrate, or a value between any of the preceding numbers, e.g., between 20% and about 25%, between about 25% and about 30%, or the like.

In some embodiments, the drape seal subsection may have a length of about 0.07 m to about 0.15 m. For example, the drape seal subsection may have a length in m of about 0.07, 0.08, 0.09, 0.10, 0.11, 0.12, 0.13, 0.14, or 0.15, or a value between any of the preceding numbers, e.g., between about 0.9 and about 0.12, between about 0.10 and about 0.14, or the like.

In some embodiments, the drape seal subsection in a three-piece construction may include contouring along one or more of first base AB and the base opposite first base AB. In other embodiments, the drape seal subsection may include printed markings and/or perforations for cutting such contourings.

In some embodiments, the drape seal subsection has concave contouring along first base AB. In some embodiments, the contouring may be semicircular or semielliptical in shape with a radius measurement of about 0.07 m to about 0.3 m. See, for example, FIG. 1F (126). For example, the contouring may have a radius measurement in m of about 0.07, 0.08, 0.09, 0.10, 0.12, 0.14, 0.16, 0.18, 0.20, 0.22, 0.24, 0.26, 0.28, or 0.30, or a value between any of the preceding numbers, e.g., between about 0.10 and about 0.20, between about 0.14 and about 0.24, or the like.

In some embodiments, the drape seal subsection has convex contouring along the base opposite first base AB. In some embodiments, the contouring may be semicircular or semielliptical in shape with a radius measurement of about 0.07 m to about 0.15 m. See, for example, FIG. 2E (234). For example, the contouring may have a radius measurement in m of about 0.07, 0.08, 0.09, 0.10, 0.11, 0.12, 0.13, 0.14, or 0.15, or a value between any of the preceding numbers, e.g., between about 0.08 and about 0.12, between about 0.13 and about 0.15, or the like.

In some embodiments, the surgical incise seal subsection may extend from about 33% to about 50% of the maximum length of the flexible substrate. For example, the surgical incise seal subsection may extend from about 33%, 34%, 35%, 36%, 37%, 38%, 39%, 40%, 41%, 42%, 43%, 44%, 45%, 46%, 47%, 48/c, 49%, or 50% of the maximum length of the flexible substrate, or a value between any of the preceding numbers, e.g., between about 35% to about 40%, between about 40% and about 45%, or the like.

In some embodiments, the surgical incise seal subsection may have a length of about 0.2 m to about 0.3 m. For example, the surgical incise seal subsection may have a length in m of about 0.20, 0.21, 0.22, 0.23, 0.24, 0.25, 0.26, 0.27, 0.28, 0.29, or 0.30, or a value between any of the preceding numbers, e.g., between about 0.20 and about 0.25, between about 0.25 and about 0.28, or the like.

In some embodiments, the surgical incise seal subsection in a three-piece construction may include contouring along a surface proximate the drape seal subsection and/or a surface proximate the stockinette seal subsection. In other embodiments, the surgical incise seal subsection may include printed markings and/or perforations for cutting such contourings.

In some embodiments, the surgical incise seal subsection in a three-piece construction may include convex contouring along a surface proximate the drape seal subsection. In some embodiments, the contouring may be semicircular or semielliptical in shape with a radius measurement of about 0.07 m to about 0.15 m. See, for example, FIG. 2E (236). For example, the contouring may have a radius measurement in m of about 0.07, 0.08, 0.09, 0.10, 0.11, 0.12, 0.13, 0.14, or 0.15, or a value between any of the preceding numbers, e.g., between about 0.08 and about 0.12, between about 0.13 and about 0.15, or the like.

In some embodiments, the stockinette seal subsection may extend from second base CD to about 25% to about 33% of the maximum length of the flexible substrate. For example, the stockinette seal subsection may extend from the second base CD to about 25%, 26%, 27%, 28%, 29%, 30%, 31%, 32%, or 33%, or a value between any of the preceding numbers, e.g., between about 25% and about 30%, between about 28% and about 32%, or the like.

In some embodiments, the stockinette seal subsection may have a length of about 0.15 m to about 0.25 m. For example, the stockinette seal subsection may have a length in m of about 0.15, 0.16, 0.17, 0.18, 0.19, 0.20, 0.21, 0.22, 0.23, 0.24, or 0.25, or a value between any of the preceding numbers, e.g., between about 0.15 and about 0.20, between about 0.20 and about 0.25, or the like.

In some embodiments, the stockinette seal subsection in a three-piece construction may include contouring along one or more of second base CD and the base opposite first base CD. In other embodiments, the stockinette seal subsection may include printed markings and/or perforations for cutting such contourings.

In some embodiments, the stockinette seal subsection in a three-piece construction may include convex contouring along second base CD. In some embodiments, the contouring may be semicircular or semielliptical in shape with a radius measurement of about 0.07 m to about 0.15 m. See, for example, FIG. 2D (230). For example, the contouring may have a radius measurement in m of about 0.07, 0.08, 0.09, 0.10, 0.11, 0.12, 0.13, 0.14, or 0.15, or a value between any of the preceding numbers, e.g., between about 0.08 and about 0.12, between about 0.13 and about 0.15, or the like.

### Characteristics of Bases AB and CD

In some embodiments, first base AB and second base CD may have a AB_{L}:CD_{L} ratio of at least about 2:1. For example, the AB_{L}:CD_{L} ratio may be about 2:1 to about 7:1. For example, the AB_{L}:CD_{L} ratio may be selected from about 2:1, 2.5:1, 3:1, 3.5:1, 4:1, 4.5:1, 5:1, 5.5:1, 6:1, 6.5:1, and 7:1, or a value within a range between any of the preceding values, e.g., between about 2:1 and about 4:1, between about 3:1 and about 5:1, or the like.

In many embodiments, the first base AB and second base CD dimensions (i.e., first base length AB_{L} and second base length CD_{L}) may be independently selected in any combination from the values listed below, provided that AB_{L}>CD_{L}.

### First Base AB

In other embodiments, first base AB may have a curvature with respect to a straight line measured between vertices and A and B. For example, first base AB may have a parabolic curvature configured to drape around a neck portion.

In some embodiments, the first base AB may have a first base length AB_{L} selected from about 0.15 m to about 0.65 m. For example, the first base AB may have a first base length AB_{L}, in m of about 0.15, 0.20, 0.25, 0.30, 0.35, 0.40, 0.45, 0.50, 0.55, 0.60, and 0.65, or a value within a range between any of the preceding values, e.g., between about 0.20 and about 0.40, between about 0.35 and about 0.60, or the like.

In some embodiments, the first base AB may include contouring as described in more detail below.

### Second Base CD

In some embodiments, the second base CD may have a second base length CD_{L} selected from about 0.07 m to about 0.3 m. For example, the second base CD may have a second base length CD_{L}, in m of about 0.07, 0.1, 0.12, 0.14, 0.16, 0.18, 0.2, 0.22, 0.24, 0.26, 0.28, and 0.3, or a value within a range between any of the preceding values, e.g., between about 0.14 and about 0.22, between about 0.12 and about 0.26, or the like.

In some embodiments, the second base CD may include contouring as described in more detail below.

### AB/CD Contouring

In some embodiments, one or more of first base AB and second base CD may include contouring. The contouring may be concave, convex, or a combination thereof.

In some embodiments, first base AB may include concave contouring designed to traverse the area over the clavicle and trapezius muscle. In some embodiments, the contouring may be semicircular or semielliptical in shape with a radius measurement of about 0.07 m to about 0.6 m. See, for example, FIG. 1F. The omitted surface area of the concave contouring may be about less than 3% of the total flexible substrate surface area. For example, the contouring may have a radius measurement in m of about 0.07, 0.08, 0.09, 0.10, 0.12, 0.14, 0.16, 0.18, 0.20, 0.22, 0.24, 0.26, 0.28, 0.30, 0.32, 0.34, 0.36, 0.38, 0.40, 0.42, 0.44, 0.46, 0.48, 0.50, 0.52, 0.54, 0.56, 0.58, or 0.60, or a value between any of the preceding numbers, e.g., between about 0.08 and about 0.12, between about 0.13 and about 0.15, or the like.

In some embodiments, second base CD may include concave contouring designed to encircle the wrist. The contouring may be semicircular or semielliptical in shape with a radius measurement of about 0.07 m to about 0.15 m. See, for example, FIG. 2D. The omitted surface area of the concave contouring may be about less than 2% of the total flexible substrate surface area. For example, the contouring may have a radius measurement in m of about 0.07, 0.08, 0.09, 0.10, 0.11, 0.12, 0.13, 0.14, or 0.15, or a value between any of the preceding numbers, e.g., between about 0.08 and about 0.12, between about 0.13 and about 0.15, or the like.

### Characteristics of Sides AC and BD

In some embodiments, first side AC and second side BD may have a AC_{L}:BD_{L} ratio of about 1:1. In some embodiments, the AC_{L}:CD_{L} ratio may be about 1:1 to about 3:1. For example, the AC_{L}:BD_{L} ratio may be selected from about 1:1, 1.1:1, 1:1.2, 1.3:1, 1.4:1, 1.5:1, 2:1, 2.5:1, or a value within a range between any of the preceding values, e.g., between about 1:1 and about 1:1.5, between about 1.5:1 and about 2:1, or the like.

In many embodiments, the first side AC and second side CD dimensions (i.e., first side length AC_{L} and second side length BD_{L}) may be independently selected in any combination from the values listed below.

### First Side AC/Second Side BD

In some embodiments, first side length AC_{L} and second side length BD_{L} may be independently selected from about 0.35 m to about 0.75 m. For example, the first side length AC_{L} and the second side length BD_{L} may independently selected, in m, of about 0.35, 0.4, 0.45, 0.5, 0.55, 0.6, 0.65, 0.7, and 0.75, or a value within a range between any of the preceding values, e.g., between about 0.4 and about 0.6, between about 0.5 and about 0.7, or the like.

### AC/BD Contouring

In some embodiments, one or more of first side AC and second side BD may include contouring. The contouring may be concave, convex, or a combination thereof.

In some embodiments, one or more of first side AC and second side BD may include concave contouring designed to allow for the surgical incise drape to conform around the curvature of the shoulder. The contouring may be located at about 20% to about 33% of the side length measured from vertices A and/or B. For example, the contouring may be located at about 0.07 m to about 0.15 m from vertices A and/or B along the respective side. In some embodiments, the contouring may have a depth or local minimum of about 0.01 m to about 0.06 m. For example, the contouring may have depth or local minimum, in m, of about 0.01, 0.015 0.02, 0.025, 0.03, 0.035, 0.04, 0.045, 0.05, 0.055, or 0.06, or a value within a range between any of the preceding numbers, e.g., between about 0.025 and about 0.05, between about 0.03 and about 0.04, or the like. The concave contouring may conceptually separate the region defined as the drape seal and the region defined as the surgical incise seal. See, for example, FIG. 1F.

In some embodiments, one or more of first side AC and second side BD may include concave contouring designed to allow for the surgical incise drape to conform around the curvature of the elbow. The contouring may be located at about 25% to about 33% of the side length measured from vertices C and/or D. For example, the contouring may be located at about 0.20 m to about 0.25 m from vertices C and/or D along the respective side. In some embodiments, the contouring may have a depth or local minimum of about 0.01 m to about 0.05 m. For example, the contouring may have depth or local minimum, in m, of about 0.01, 0.015 0.02, 0.025, 0.03, 0.035, 0.04, 0.045, or 0.05, or a value within a range between any of the preceding numbers, e.g., between about 0.025 and about 0.05, between about 0.03 and about 0.04, or the like. The concave contouring may conceptually separate the region defined as the surgical incise seal and the region defined as the stockinette seal. See, for example, FIG. 2D.

Characteristics of Angles A_{θ}, B_{θ}, C_{θ}, and D_{θ}

As used herein, angle A_{θ} is defined as the angle formed between first base AB and first side AC, angle B_{θ} is defined as the angle formed between first base AB and second side BD, angle C_{θ} is defined as the angle formed between first side AC and second base CD, and angle D_{θ} is defined as the angle formed between second base CD and second side BD.

In some embodiments, A_{θ}=B_{θ}.

In some embodiments, C_{θ}=D_{θ}.

In some embodiments, A_{θ}=B_{θ} and C_{θ}=D_{θ} and A_{θ}≠C_{θ}.

In some embodiments, A_{θ}<B_{θ}.

In some embodiments, C_{θ}>D_{θ}.

In some embodiments, A_{θ}<B_{θ} and C_{θ}>D_{θ} and A_{θ}≠D_{θ}.

In many embodiments, angles A_{θ}, B_{θ}, C_{θ}, and D_{θ}, and any combination thereof, may be selected from the values described below.

Angle A_{θ}/Angle B_{θ}

In some embodiments, angles A_{θ} and angle B_{θ} may be independently selected from about 75° to about 89°. For example, angle A_{θ} and angle B_{θ} may be independently selected from about 75°, 76°, 77°, 78°, 79°, 80°, 81°, 82°, 83°, 84°, 85°, 86°, 87°, 88°, and 89°, or a value within a range between any of the preceding values, e.g., between about 80° to about 85°, between about 77° and about 87°, or the like.

Angle C_{θ}/Angle D_{θ}

In some embodiments, angle C_{θ} and angle D_{θ} may be independently selected from about 100° to about 105°. For example, angle C_{θ} angle D_{θ} may be independently selected from about 100°, 101°, 102°, 103°, 104°, and 105°, or a value within a range between any of the preceding values, e.g., between about 100° to about 102°, between about 101° and about 104°, or the like.

### Antimicrobial Adhesive Compositions

In many embodiments, the antimicrobial adhesive composition may include one or more antimicrobial agent described below and one or more pressure-sensitive adhesive described below.

In some embodiments, the antimicrobial adhesive composition may consist essentially of one or more antimicrobial agent described below and one or more pressure-sensitive adhesive described below.

In some embodiments, the antimicrobial agent is completely dissolved within the pressure-sensitive adhesive. In other embodiments, the antimicrobial agent is at least partly suspended within the pressure-sensitive adhesive. The antimicrobial agent should readily diffuse through the pressure-sensitive adhesive so as to supply the surface of the composition, and therefore the skin surface in contact with the composition, with the antimicrobial agent in order to continually disinfect the skin surface.

In some embodiments, the antimicrobial adhesive composition may further include one or more of a plasticizer, a thickener, a tackifier, a stabilizer, an initiator, a crosslinking agent, a solubilizing agent (e.g., monoacylglyceride), a viscosity modifier, a surfactant (e.g., nonionic, cationic, anionic, zwitterionic surfactant), a moisturizing agent, and a vitamin and/or mineral. In some embodiments, the antimicrobial adhesive composition may further include agents to improve stability, viscosity, adhesion, or the like.

### Antimicrobial Agents

Generally, the antimicrobial adhesive composition may include any antimicrobial agent, i.e., a chemical compound or complex, known to a skilled artisan, that has been demonstrated to kill infectious organisms (e.g., Propionibacterium acnes), or otherwise prevent or slow the reproduction thereof. Antimicrobial agents may include antiseptics, antibacterials, antibiotics, antivirals, antifungals, antiparasitics, or the like.

In some embodiments, the antimicrobial adhesive composition may include an antimicrobial agent selected from iodine, an iodophor, a triiodide complex, chlorhexidine gluconate, chlorhexidine acetate, octenidine hydrochloride, octenidine alexidine, polymeric biguanides (e.g., polyhexamethylene biguanide), silver, a silver salt (e.g., silver chloride, silver oxide), a copper salt, a polyquaternium, a phenol (e.g., parachlorometaxylenol (PCMX), triclosan, hexachlorophene), fatty acid monoesters (e.g., glycerol monolaurate), hydrogen peroxide, and a combination thereof.

In some embodiments, the antimicrobial adhesive composition may include an antimicrobial agent selected from iodine, an iodophor, chlorhexidine gluconate, octenidine hydrochloride, polyhexamethylene biguanide, a silver salt, a copper salt, and a combination thereof.

In some embodiments, the antimicrobial agent is iodine or an iodophor (e.g., povidone iodine).

In some embodiments, the antimicrobial agent is chlorhexidine gluconate.

In some embodiments, the antimicrobial agent is octenidine hydrochloride.

In some embodiments, the antimicrobial agent is polyhexamethylene biguanide.

### Pressure-Sensitive Adhesives

Generally, the antimicrobial adhesive composition may include any pressure-sensitive adhesive, known to a skilled artisan, that has been shown to be suitable and effective in adhering to skin.

In some embodiments, the pressure-sensitive adhesive may be selected from acrylates, polyurethanes, polyvinyl ethyl ethers, silicones, rubber-based adhesives (e.g., natural rubber, polyisoprene, polyisobutylene, butyl rubber), a combination thereof, and a copolymer thereof. For example, the pressure-sensitive adhesive may include polymers available from Shell Chemical Company (Houston, TX) under the name KRATON.

In some embodiments, the pressure-sensitive adhesive is selected from an acrylic polymer or copolymer. In some embodiments, the acrylic polymer or copolymer may be the reaction product one of monomers selected from an alkyl (meth)acrylate, N-vinyl pyrrolidone, N-vinyl caprolactam, (alkyl-substituted)acrylamide, (alkyl-substituted)methacrylamide, 2-hydroxyethyl (meth)acrylate or and a combination thereof.

In some embodiments, the pressure-sensitive adhesive may be characterized by a glass transition temperature (T_{g}) of about −70° C. to about 20° C. In some embodiments, the pressure-sensitive adhesive may be characterized by a T_{g} in ° C. of about −70, −60, −50, −40, −30, −20, −10, −5, 0, 5, 10, or 20, or a value within a range between any of the preceding values, for example, between about −20 and about 5, between about −50 and about −30, or the like.

### Delivery Systems

A delivery system may provide additional stability to the flexible substrate. Surgical incise drapes having a delivery system may mitigate wrinkling upon applying the surgical incise drape to a skin surface. A delivery system may further assist in placement of the surgical incise drape. The delivery system may be removed in sections to further assist delivery.

The delivery system may be constructed from a material more rigid than that of the flexible substrate. For example, the delivery system may include one or more materials selected from polyethylene/vinyl acetate copolymer, polyvinylacetate, polyester film, polyethylene film, nonwovens, polymer films, and papers. The delivery system may be releasably secured to the flexible substrate with a suitable adhesive.

In some embodiments, the delivery system may overlay at least a portion of the flexible substrate. For example, the delivery system may overlay about 10% to about 100% of the surface area of the flexible substrate. For example, the delivery system may overlay (in surface area %) about 10, 15, 20, 25, 30, 35, 40, 45, 50, 55, 60, 65, 70, 75, 80, 85, 90, 100, or a value within a range between any of the preceding values, e.g., between about 15 and about 40, between about 60 and about 80, or the like.

In some embodiments, the delivery system may include a perimeter liner, i.e., a material adhered to the perimeter of the surgical incise drape. Perimeter liners may be comprised of one or more pieces.

In some embodiments, the delivery may include a surface liner, i.e., a material adhered to at least a portion of the surface of the surgical incise drape. Surface liners may be configured as a sheet or may be of a patterned configuration. Surface liners may be comprised of one or more pieces.

In some embodiments, the delivery system may include a perimeter liner and a surface liner. In some embodiments, the material of the perimeter liner and the surface liner may be the same or different.

In some embodiments, the delivery system may include one or more perforations that may assist in sectioned removal of the delivery system. Perforations may be partial cuts or other means of dividing a material into sections.

In some embodiments, the delivery system may include a surface liner extending over more than 80% of the flexible substrate surface. The surface liner may be perforated or otherwise divided into sections. In some embodiments, the surface liner may include three sections. In some embodiments, the surface liner may have one or more perforations extending from the first base to the second base. In other embodiments, the surface liner may have one or more perforations extending from the first side to the second side.

In some embodiments, the delivery may include one or more handling tabs. The handling tabs may extend beyond the perimeter of the flexible substrate and/or above the surface of the flexible substrate. Handling tabs may assist a user in appropriately placing the surgical incise drapes, or sections thereof, to a skin surface.

### Release Liners

The release liner may include or be a kraft paper, a glassine paper, polyethylene, polypropylene, polyester, and a combination thereof.

In some embodiments, the release liner may be perforated. Perforations in the release liner may allow for the application of the surgical incise drape in sections. In some embodiments, the surface liner may have one or more perforations extending from the first base to the second base. In other embodiments, the surface liner may have one or more perforations extending from the first side to the second side.

### Methods of Preparation

In one embodiment, a method of making a trapezoidal surgical incise drape is described. The method may include providing a flexible substrate and an antimicrobial adhesive composition; and disposing the antimicrobial adhesive composition onto one or more of the flexible substrate.

In some embodiments, the method may further include providing a release liner and contacting the release liner to the antimicrobial adhesive composition.

In some embodiments, the method may further include providing a delivery system and adhering the delivery system to a surface of the flexible substrate that is opposite the surface having the antimicrobial adhesive composition disposed thereon.

### Methods of Using

In many embodiment, a method for preparing a shoulder for surgery is described. The method may include providing a trapezoidal surgical incise drape described herein; and contacting the surgical incise drape to a skin surface of a subject.

In some embodiments, the skin surface may include a portion of the neck, the shoulder, and the surfaces therebetween. The skin surface may include the shoulder, the upper arm (e.g., bicep and tricep), and the surfaces therebetween. The skin surface may include the forearm, the elbow, the wrist, and the surfaces therebetween. The skin surface may include the armpit area, the under the upper arm, and the surfaces therebetween.

In some embodiments, the method may further include providing one or more rectangular surgical incise drapes and contacting the one or more rectangular surgical incise drapes to the skin surface and to a portion of the trapezoidal surgical incise drape. Placement of the rectangular surgical incise drape secures the trapezoidal surgical incise drape to the skin surface. The rectangular incise drape may only differ from the trapezoidal surgical incise drapes described herein by shape. The rectangular incise drapes may be wrapped around one or more portions of any arm structure. Rectangular surgical incise drapes may be cut to any desired size.

In some embodiments, a surgical incise drape is first applied to the axilla region. Next, a second surgical incise drape, or the drape seal subsection thereof, is attached to the top of the subject's shoulder. Following afterward, the surgical incise subsection is applied to the subject's upper arm area and then the forearm and wrist. In some embodiments, a middle portion of a release liner is then removed upon anchoring the drape to the skin, following by removal of left and right release liner portions prior to wrapping the drape around the arm.

### Kits

In various embodiments, a kit is described. The kit may include one or more surgical incise drapes described herein and a set of instructions directed a user to perform method steps for preparing a shoulder for surgery described herein.

In some embodiments, the kit may include a one-piece surgical incise drape described herein.

In some embodiments, the kit may include a three-piece surgical incise drape described herein.

In some embodiments, the kit may further include one or more rectangular surgical incise drapes described herein.

In some embodiments, the set of instructions may further direct a user in how to optimally apply the trapezoidal surgical incise drape to the skin surface.

1. A surgical incise drape comprising:
a flexible substrate having a trapezoidal configuration characterized by four vertices A, B, C, and D,
the flexible substrate comprising:
a first base (AB) defined by a first base length (AB_{L}) measured between vertices A and B,
a second base (CD) defined by a second base length (CD_{L}) measured between vertices C and D,
wherein AB_{L}>CD_{L},
a first side (AC) defined by a first side length (AC_{L}) measured between vertices A and C, and
a second side (BD) defined by a second side length (BD_{L}) measured between vertices B and D; and
an antimicrobial adhesive composition comprising:
an antimicrobial agent, and
a pressure-sensitive adhesive.

2. A surgical incise drape comprising:
a flexible substrate having a trapezoidal configuration characterized by four vertices A, B, C, and D,
the flexible substrate comprising:
a first base (AB) defined by a first base length (AB_{L}) measured between vertices A and B,
a second base (CD) defined by a second base length (CD_{L}) measured between vertices C and D,
wherein AB_{L}>CD_{L},
a first side (AC) defined by a first side length (AC_{L}) measured between vertices A and C, and
a second side (BD) defined by a second side length (BD_{L}) measured between vertices B and D; and
an antimicrobial adhesive composition comprising:
an antimicrobial agent, and
a pressure-sensitive adhesive,
wherein the flexible substrate is divided into three separate subsections, and
wherein the three separate subsections together form the trapezoidal configuration.

3. The surgical incise drape of claim 1, wherein the flexible substrate comprises a polyurethane film, a polyester film, a polyether block amide film, a thermoplastic polyester elastomer film, or a combination thereof.

4. (canceled)

5. The surgical incise drape of claim 1, wherein the flexible substrate has a thickness of about 25 μm to about 200 μm.

6. The surgical incise drape of claim 1, the flexible substrate characterized having a length from about 0.5 m to about 0.7 m.

7. The surgical incise drape of claim 1, wherein the ratio AB_{L}:CD_{L} is at least about 2:1.

8. (canceled)

9. The surgical incise drape of claim 1, wherein AB_{L} is selected from about 0.15 m to about 0.65 m.

10. The surgical incise drape of claim 1, wherein CD_{L} is selected from about 0.07 m to about 0.3 m.

11. The surgical incise drape of claim 1, wherein the ratio AC_{L}:BD_{L} is 1:1.

12. The surgical incise drape of claim 1, wherein AC_{L} and BD_{L} are independently selected from about 0.35 m to about 0.75 m.

13. The surgical incise drape of claim 1, wherein AB and AC form an angle A_{θ}, and AB and BD form an angle B_{θ}, wherein angle A_{θ} and angle B_{θ}, are independently selected from about 75° to about 89°.

14. The surgical incise drape of claim 1, wherein AC and CD form an angle C_{θ} and CD and BD form an angle D_{θ}, wherein angle C_{θ} and angle D_{θ} are selected from about 100° to about 105°.

15. The surgical incise drape of claim 1, wherein one or more of the first base AB, the second base CD, the first side AC, and the second side BD comprise concave contouring.

16. (canceled)

17. The surgical incise drape of claim 1, wherein the antimicrobial agent is selected from iodine, an iodophor, chlorhexidine gluconate, octenidine hydrochloride, polyhexamethylene biguanide, a silver salt, a copper salt, and a combination thereof.

18. The surgical incise drape of claim 1, wherein the pressure-sensitive adhesive is selected from acrylates, polyurethanes, silicones, rubber-based adhesives, and a combination thereof.

19. The surgical incise drape of claim 1, further comprising a delivery system in contact with at least a portion of the flexible substrate.

20. The surgical incise drape of claim 19, wherein the delivery system comprises handling tabs.

21. (canceled)

22. (canceled)

23. (canceled)

24. (canceled)

25. A method of preparing a surgical incise drape of claim 1, the method comprising:
providing a flexible substrate- and an antimicrobial adhesive composition; and
disposing the antimicrobial adhesive composition onto the flexible substrate.

26. (canceled)

27. (canceled)

28. A method for preparing a shoulder for surgery, the method comprising:
providing a surgical incise drape of claim 1; and
contacting the surgical incise drape to a skin surface of a subject.

29. (canceled)

30. A kit comprising:
a surgical incise drape of claim 1; and
a set of instructions directing a user to contact the surgical incise drape to a skin surface of a subject.

31. (canceled)
